(19) United States
(12) Patent Application Publication
(10) Pub. No.: US 2005/0053671 A1
(43) Pub. Date: Mar. 10, 2005

(54) Alternate soaking apparatus and method for treatment of tissue materials

(75) Inventors: Junzo Tanaka, Ibaraki (JP); Masataka Sakane, Ibaraki (JP); Hironobu Fukuzaki, Hyogo (JP); Yoshikane Yokoyama, Tokyo (JP); Kunihiro Ota, Tokyo (JP); Masanori Kikuchi, Ibaraki (JP); Toshiyuki Ikoma, Ibaraki (JP); Isamu Yamaguchi, Ibaraki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: JAPAN SCIENCE AND TECHNOLOGY AGENCY, Kawaguchi-shi (JP); NATIONAL INSTITUTE FOR MATERIALS SCIENCE, Tsukuba-shi (JP); TAMACHI INDUSTRIES CO., LTD., Tokyo (JP); TAKI CHEMICAL CO., LTD., Kakogawa-shi (JP); ESCOM CO., LTD., Tokyo (JP)

(21) Appl. No.: 10/927,054

(22) Filed: Aug. 27, 2004

Related U.S. Application Data
(63) Continuation of application No. PCT/JP02/07576, filed on Jul. 25, 2002.

(51) Int. Cl. A61K 33/42

(52) U.S. Cl. 424/602

(57) ABSTRACT

The alternate soaking apparatus for treatment of tissue materials has a plurality of chemical liquid containers, a treatment container for soaking and treating a tissue material, a plurality of liquid feed tubes for providing fluid communication between the corresponding chemical liquid containers and the treatment container to introduce the respective chemical liquids into the treatment container, a liquid discharge tube for discharging the chemical liquids in the treatment container, and control means for controllably opening and closing the liquid feed tubes and the liquid discharge tube to allow the plurality of chemical liquids to be contained in the treatment container individually and alternately. A tissue material is treated using the above alternate soaking apparatus under the condition that one of chemical liquids is an aqueous solution containing calcium, and another one of chemical liquids is an aqueous solution containing phosphoric acid, so as to form a calcium phosphate compound on the surface of and in the inside of the treated tissue material. The present invention allows a ligament material and to be automatically synthesized with a calcium phosphate-based compound in a shorter time-period by a limited number of members during the operation while securing an aseptic condition.

### TECHNICAL FIELD

The present invention relates to an apparatus and a method for treatment of the surface and inside of a tissue material, such as natural or artificial soft tissue materials including a ligament or tendon material, in preparing for implantation thereof. In particular, the present invention relates to an alternate soaking apparatus and method for forming a calcium phosphate-based compound on the surface of and in the inside of a tissue material.

### BACKGROUND ART

There have been known various methods for coating the surface of biomaterials, such as bone-repairing materials, artificial organs or implantable medical apparatus, with a hydroxyapatite layer having a structure and composition analogous to a bone. Japanese Patent Publication No. H06-29126 discloses one related method of coating a base material with an active hydroxyapatite layer by soaking the base material in an aqueous solution containing hydroxyapatite at a saturated concentration. Japanese Patent Laid-Open Publication No. H06-327757 discloses another method of coating an organic or inorganic fiber aggregation with a calcium phosphate-based compound by a liquid-phase deposition process.

Japanese Patent laid-Open Publication No. 2001-254264 discloses still another method of producing a hydroxyapatite-fiber complex, comprising steps of irradiating a fiber material with an electron beam to graft-polymerize a vinyl monomer thereonto, and alternately soaking the fiber material in an aqueous solution of calcium chloride and an aqueous solution of disodium hydrogen phosphate to form hydroxyapatite thereon.

A transplantation of an artificial or autologous ligament has been utilized in a medical treatment of repairing a damaged ligament. In this case, it is required to allow the artificial or autologous ligament to adhere directly to a bone.

However, the conventional repairing treatment techniques/technologies involve a problem, such as avulsion or shear fracture of the ligament due to insufficient bonding strength. As one of measures for solving this problem, the inventors developed an effective alternate soaking method for reforming a soft tissue material, such as a tendon or ligament material, and applied for a patent (Japanese Patent Application No. H11-323753, Japanese Patent Laid-Open 2001-137329).

Specifically, the above method for reforming a soft tissue material is a technique of synthesizing a matrix (soft tissue material) containing collagen as a principal component and a calcium phosphate-based compound containing hydroxyapatite which has a biocompatibility, cell-differentiation/induction ability and bone-conduction/induction ability. This method can provide a reformed soft tissue material, such as a ligament or tendon material, which has a high affinity to a bone as a hard tissue, and an improved bonding rate/strength with the bone after a reconstructive surgery.

In the practical application of the alternate soaking method at an operation site, it is required to synthesizing a ligament material and a calcium phosphate-based compound in a shorter time-period by a limited number of members during the operation. Thus, there is the need for automatically performing the method while securing an aseptic condition.

### SUMMARY OF THE INVENTION

Through previous researches on molecule complex systems, the inventors verified that a tendon or ligament material can be synthesized with a calcium phosphate-based compound, as disclosed in the aforementioned Japanese Patent Application No. H11′-323753 (Japanese Patent Laid-Open 2001-137329).

The specification of the above patent application specifically discloses a method for forming a calcium phosphate-based compound containing hydroxyapatite, in the surface layer of a tendon or ligament material, comprising the steps of soaking a tendon and/or ligament materials in an aqueous solution containing calcium ions (at a concentration of 0.1 to 40 wt %), then soaking the materials in distilled water or physiological saline, then soaking the material in an aqueous solution containing phosphate ions (at a concentration of 0.1 to 20 wt %), wherein the above steps are performed one time or repeated two or more times.

This method can provide a complex material obtained improved in the bonding between a ligament or tendon material as a soft tissue and a bone as a hard tissue, which has caused problems in operations, such as in anterior cruciate ligament reconstruction, using an autologous tissue.

Through further researches on an apparatus for synthesizing a tissue material and a calcium phosphate-based compound automatically and aseptically using an alternate soaking method, with a view of meeting the above need, the inventors have finally accomplished an alternate soaking apparatus and method suitable for treatment of the surfaces of natural or artificial tissue materials, such as soft tissue materials including a ligament or tendon material.

Specifically, the present invention provide an alternate soaking apparatus for treatment of tissue materials, comprising a plurality of chemical liquid containers each for containing a different chemical liquid, a treatment container for soaking and treating a tissue material therein, a plurality of liquid feed passages for providing fluid communication between the corresponding chemical liquid containers and the treatment container to introduce the respective chemical liquids into the treatment container, a liquid discharge passage for discharging the chemical liquids in the treatment container, and control means for controllably opening and closing the liquid feed passages and the liquid discharge passage to allow the plurality of chemical liquids to be contained in the treatment container individually and alternately.

In the alternate soaking apparatus, the control means may include a plurality of valves provided in the corresponding liquid feed passages and the liquid discharge passage, and an automatic controller for automatically controlling the valves between their open and close states.

In the alternate soaking apparatus, the treatment container may include a top cover provided with a hook for fixing the tissue material.

In the alternate soaking apparatus, the treatment container may have a sidewall formed with an insertion hole for allowing the tissue material to be put into the treatment container from outside therethrough.

The present invention also provides an alternate soaking method for treatment of tissue materials. In this method, a tissue material is treated using the above alternate soaking apparatus, wherein one of the plurality of chemical liquids is an aqueous solution containing calcium, and another one of the plurality of chemical liquids is an aqueous solution containing phosphoric acid, whereby a calcium phosphate compound is formed on the surface of and in the inside of the treated tissue material.

According to the automatized alternate soaking apparatus of the present invention, a calcium phosphate-based compound can, for example, be formed on the surface of and in the inside of a tissue material, such as a ligament or tendon material, by placing the tissue material in an aseptic container and automatically filling the aseptic container with an aqueous solution containing phosphoric acid and an aqueous solution containing calcium alternately without mixture thereof.

The tissue material may include: a hydrophilic soft tissue material, such as a ligament or tendon material; and as artificial tissue material, such as chitin, chitosan, fluorocarbon resin typified by Gore-Tex (registered trademark), polyester, polylactic acid, polyvinyl alcohol, collagen, gelatin, chondroitin sulfate, hyaluronic acid and a combination thereof. Among them, some tissue materials, such as collagen, gelatin, chondroitin sulfate and hyaluronic acid, often have water solubility. In this case, it is required to gel them, for example, through cross-linking.

The calcium phosphate-based compound to be formed on the surface of and in the inside of the tissue material may include amorphous calcium phosphate, hydroxyapatite, octacalcium phosphate, tricalcium phosphate and dicalcium phosphate dihydrate.

### BRIEF DESCRIPTION OF THE DRAWINGS

FIG. 1 is a schematic side view showing an alternate soaking apparatus according to one embodiment of the present invention.

FIG. 2 is a schematic side view showing one example of a treatment container for soaking a tissue material therein, in the alternate soaking apparatus of the present invention.

FIG. 3 is a schematic side view showing another example of the treatment container in the alternate soaking apparatus of the present invention.

### BEST MODE FOR CARRYING OUT THE INVENTION

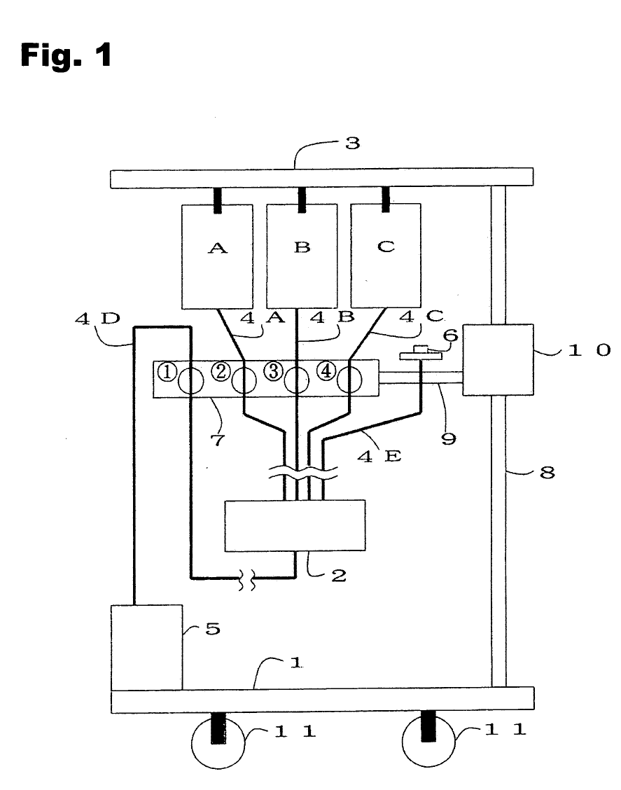

With reference to FIGS. 1 to 3, an alternate soaking apparatus according to one embodiment of the present invention will now be described.

As shown in FIG. 1, a single treatment container 2 for soaking a tissue material therein is attached to an I.V. stand 1, and three I.V. or drip bags A, B, C serving as a plurality of chemical liquid containers are hung from a hanging bar 3 provided on the top of the I.V. stand. Preferably, the I.V. stand 1 is configured to be movable by providing wheels 11 thereto. Three liquid feed tubes 4A, 4B, 4C are provided in such a manner that ones of the ends of the liquid feed tubes are connected, respectively, to the drip bags A, B, and C, and each of the other ends thereof is connected to the treatment container 2 to provide fluid communication between the drip bags A, B, C and the treatment container 2.

Further, a liquid discharge tube 4D is provided in such a manner that one of the ends of the discharge tube is connected to the bottom of the treatment container 2, and the other end thereof is connected to a vacuum bottle 5 which is mounted on the I.V. stand 1 to receive the liquid discharged from the treatment container 2, so as to provide fluid communication between the treatment container 2 and the vacuum bottle 5.

An air tube 4E is also attached to the treatment container 2 to introduce outside air into the treatment container 2 during a discharge operation of discharging the liquid from the treatment container 2 into the vacuum bottle 5. The air tube 4E is necessary to maintain the inner pressure of the treatment container 2 at atmosphere pressure during the discharge operation. Preferably, the air tube 4E is provided with an air filter 6 for cleaning air introduced into the treatment container 2, at one end thereof opened to atmospheric air.

Each of the liquid feed tubes 4A, 4B, and 4C and the liquid discharge tube 4D is provided with control means interposed therein to controllably open and close the respective tubes. The control means may comprise a valve assembly 7 integrally including first, second, third and forth valves 1, 2, 3, 4 provided in the liquid discharge tube 4D, the liquid feed tube 4A, the liquid feed tube 4B and the liquid feed tube 4C, respectively. The valve assembly 7 may be supported horizontally by a support bar 9 attached to a column support 8 of the I.V. stand

Each of the valves is not limited to a specific structure or type, but any suitable valve capable of selectively blocking the flow of the liquid in the tube and allowing the liquid to flow therethrough may be used. In one preferred embodiment, the tube has flexibility, and the valve may be a type of selectively pinching and choking the resilient tube to control the flow of the liquid.

Each of the first to fourth valves 1, 2, 3, 4 is controllably opened and closed by programmed automatic control signals from a valve controller 10 attached at an appropriate position of the I.V. stand. The liquid feed tubes 4A, 4B, and 4C are selectively opened and closed by the second, third and fourth valves 2, 3, 4, respectively. The liquid discharge tube 4D is selectively opened and closed by the first valve 1.

Preferably, the treatment container 2 is made of polyvinylchloride. The use of polyvinylchloride is advantageous to allow the treatment container 2 to be subjected to a sterilization process using a gas (ethylene oxide) as used in drip tubes. The treatment container 2 is fundamentally disposable. It is understood that the material of the treatment container 2 is not limited to polyvinylchloride, but any other suitable material capable of being subjected to a sterilization process and being disposable. The liquid feed tube may be a commonly used drip tube.

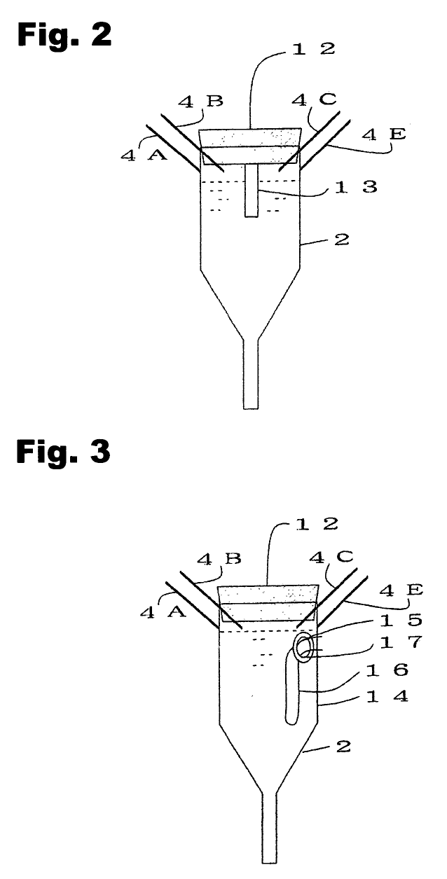

FIG. 2 shows one example of the treatment container 2 for use if a tissue material to be treated can be freely moved (such as an isolated ligament or tendon material separated from a living body). In this example, the treatment container 2 includes a top cover 12, and a hook 13 is provided at the center of the inner surface of the top cover 12. The tissue material is fixed to the hook 13.

FIG. 3 shows another example of the treatment container 2 for use if a tissue material to be treated is not freely moved (such as one end portion of a ligament or tendon material connected with a living body). In this example, the treatment container 2 has a sidewall 14 formed with an insertion hole 15 for allowing a tissue material to be put into the treatment container 2 from outside therethrough. The treatment container 2 is moved to get close to the shoulder or knee, and a tissue material 16 or one end portion of a ligament or tendon material is put into the treatment container 2 through the insertion hole 15. If there is a certain clearance between the tissue material 16 and the insertion hole 15, the chemical liquid flowing in the treatment container 2 will leak therefrom. In order to prevent this leakage, a packing 17 is provided around the insertion hole 15.

In preparing for forming a calcium phosphate compound on the surface of and in the inside of a tissue material, a liquid A of an aqueous solution containing calcium (e.g. CaCl₂), a liquid B of an aqueous solution for washing (e.g. distilled water or saline), and a liquid C of an aqueous solution containing phosphate (e.g. sodium dihydrogen phosphate, disodium hydrogen phosphate, and trisodium phosphate) are filled in the drip bags A, B, and C, respectively.

Preferably, each of the aqueous solutions has a concentration for providing an osmotic pressure close to that of physiological saline (279 mmol/kg). An excessively high concentration would cause increased damage in the tissue material. If the concentration is excessively low, a reduced amount of calcium phosphate will cause deterioration in the formation of calcium phosphate per time. This characteristic is disadvantageous to the apparatus to be used during an operation.

A method for treatment of a tissue material using the above alternate soaking apparatus of the present invention will be described with reference to FIGS. 1 to 3.

The operation of each of the valves will be first described in conjunction with a time chart of the valve controller 10 (1 cycle). Each of the valves can be arranged to have any suitable time-period in the open or close state, and any suitable number of cycles. While a greater number of cycles provides an increased amount of a calcium phosphate-based compound to be formed, an operation time will be extended.

The valves in their close state are operated as follows by pressing a start button of the valve controller 10:
(1) The second valve 2 is opened to allow the liquid A to flow into the treatment container 2 (T1),
(2) The second valve 2 is closed, and the supplied liquid A remains in the treatment container 2 (T2),
(3) The first valve 1 is opened, and air flows into the treatment container 2 through the filter 6 and the tube 4E while discharging the liquid A from the treatment container 2 (T3),
(4) The first valve 1 is closed, and the third valve 3 is opened to allow the liquid B to flow into the treatment container 2 (T4),
(5) The third valve 3 is closed, and the supplied liquid B remains in the treatment container 2 (T5),
(6) The first valve 1 is opened, and air flows into the treatment container 2 through the filter 6 and tube 4E while discharging the liquid B from the treatment container 2 (T6),
(7) The first valve 1 is closed, and the fourth valve 4 is opened to allow the liquid C to flow into the treatment container 2 (T7),
(8) The fourth valve 4 is closed, and the supplied liquid C remains in the treatment container 2 (T8),
(9) The first valve 1 is opened, and air flows into the treatment container 2 through the filter 6 and tube 4E while discharging the liquid C from the treatment container 2 (T9),
(10) The first valve 1 is closed, and the third valve 3 is opened to allow the liquid B to flow into the treatment container 2 (T10),
(11) The third valve 3 is closed, and the supplied liquid B remains the treatment container 2 (T11),
(12) The first valve 1 is opened, and air flows into the treatment container 2 through the filter 6 and the tube 4E while discharging the liquid B from the treatment container 2 (T12), and
(13) The first valve 1 is closed.

The time-period in the open or close state of each of valves in the above operation will be specifically described below.

T1: T1 is required to have a time-period for allowing the liquid A to be filled in the treatment container.

T2: While a longer time for soaking the tissue material in the liquid A provides increased formation of the calcium phosphate-based compound, the operation time will be extended.

T3: T3 is required to have a time-period for allowing the liquid A to be completely discharged from the treatment container.

T4: T4 is required to have a time-period for allowing the liquid B to be filled in the treatment container.

T5: T5 is set to have just a time-period (about 30 seconds) for allowing ions remained in the soaked tissue material to be diffused in the washing solution.

T6: T6 is required to have a time-period for allowing the liquid B to be completely discharged from the treatment container.

T7: T7 is required to have a time-period for allowing the liquid C to be filled in the treatment container.

T8: While a longer time for soaking the tissue material in the liquid C provides increased formation of the calcium phosphate-based compound, the operation time will be extended.

T9: T9 is required to have a time-period for allowing the liquid C to be completely discharged from the treatment container.

T10: T10 is required to have a time-period for allowing the liquid B to be filled in the treatment container.

T11: T11 is set to have just a time-period (about 30 seconds) for allowing ions remained in the soaked tissue material to be diffused in the washing solution.

T12: T12 is required to have a time-period for allowing the liquid B to be completely discharged from the treatment container.

### EXAMPLE

### Example 1

A tissue material was treated using the apparatus described according to the above embodiments, under the following conditions. An Achilles' tendon of a rabbit was used as the tissue material to be treated. The treatment container 2 as shown in FIG. 2 was used, and the Achilles' tendon was fixed to the hook 13.

4.3 wt % calcium chloride aqueous solution, 0.9 wt % saline solution, and 1.65 wt % sodium dihydrogen phosphate solution were used as the liquids A, B, C, and filled in the drip bags A, B, C, respectively. The number of cycles was 3 cycles. The time-period in the open or close state of each of the valves was set as follows; T1: 20 seconds, T2: 180 seconds, T3: 20 seconds, T4: 20 seconds, T5: 20 seconds, T6: 20 seconds, T7: 20 seconds, T8: 180 seconds, T9: 20 seconds, T10: 20 seconds, T11: 20 seconds, and T12: 20 seconds.

In order to measure the amount of calcium phosphate formed through the alternate soaking, a sample dried at 120° C. was heated up to 1200° C. to decompose organic components therein (e.g. proteins and collagen), and the amount of the resultant inorganic substance (calcium phosphate) after the firing at 1200° C. was measured to determine a percentage thereof relative to 100 wt % of the initial dried sample. As a result, the amount of the inorganic substance formed synthesized with the Achilles' tendon of the rabbit was 8.3% of the dry weight. Any sample without such a treatment had a few amount of remnant (about 0.3%) after firing. Through an XRD analysis, it was proved that the inorganic substance in the reformed Achilles' tendon is a low crystalline hydroxyapatite.

### Example 2

A tissue material was treated using the apparatus described according to the above embodiments, under the following conditions. A chitosan porous material was used as the tissue material to be treated. The treatment container 2 as shown in FIG. 2 was used, and the chitosan porous material was fixed to the hook 13.

4.3 wt % calcium chloride aqueous solution, 0.9 wt % saline solution, and 1.66 wt % sodium dihydrogen phosphate were used as the liquids A, B, C, and filled in infusion bags A, B, C, respectively. The number of cycles was 3 cycles. The time-period in the open or close state of each of the valves was set as follows; T1: 20 seconds, T2: 180 seconds, T3: 20 seconds, T4: 20 seconds, T5: 20 seconds, T6: 20 seconds, T7: 20 seconds, T8: 180 seconds, T9: 20 seconds, T10: 20 seconds, T11: 20 seconds, and T12: 20 seconds.

As a result, the amount of the inorganic substance synthesized with the chitosan porous material was 16.3% of the dry weight. Through an XRD analysis, it was proved that the inorganic substance in the reformed chitosan porous material was a low crystalline hydroxyapatite.

1. An alternate soaking apparatus for treatment of tissue materials, comprising:
a plurality of chemical liquid containers each for containing a different chemical liquid:
a treatment container for soaking and treating a tissue material therein;
a plurality of liquid feed passages for providing fluid communication between said corresponding chemical liquid containers and said treatment container to introduce said respective chemical liquids into said treatment container;
a liquid discharge passage for discharging said chemical liquids in said treatment container; and
control means for controllably opening and closing said liquid feed passages and said liquid discharge passage to allow said plurality of chemical liquids to be contained in said treatment container individually and alternately.

2. The alternate soaking apparatus as defined in claim 1, wherein said control means includes a plurality of valves provided in said corresponding liquid feed passages and said liquid discharge passage, and an automatic controller for automatically controlling said valves between their open and close states.

3. The alternate soaking apparatus as defined in claim 1, wherein said treatment container includes a top cover provided with a hook for fixing said tissue material.

4. The alternate soaking apparatus as defined in claim 1, wherein said treatment container has a sidewall formed with an insertion hole for allowing said tissue material to be put into said treatment container from outside therethrough.

5. An alternate soaking method for treatment of tissue materials, wherein a tissue material is treated using the alternate soaking apparatus as defined in claim 1, wherein one of said plurality of chemical liquids is an aqueous solution containing calcium, and another one of said plurality of chemical liquids is an aqueous solution containing phosphate, whereby a calcium phosphate compound is formed on the surface of and in the inside of said treated tissue material.

6. The alternate soaking apparatus as defined in claim 2, wherein said treatment container includes a top cover provided with a hook for fixing said tissue material.

7. The alternate soaking apparatus as defined in claim 2, wherein said treatment container has a sidewall formed with an insertion hole for allowing said tissue material to be put into said treatment container from outside therethrough.

8. An alternate soaking method for treatment of tissue materials, wherein a tissue material is treated using the alternate soaking apparatus as defined in claim 2, wherein one of said plurality of chemical liquids is an aqueous solution containing calcium, and another one of said plurality of chemical liquids is an aqueous solution containing phosphate, whereby a calcium phosphate compound is formed on the surface of and in the inside of said treated tissue material.
